(19) United States
(12) Patent Application Publication
(10) Pub. No.: US 2023/0190888 A1
(43) Pub. Date: Jun. 22, 2023

(54) STABLE LIPASE FORMULATIONS AND METHODS THEREOF

(71) Applicant: First Wave BioPharma, Inc., Boca Raton, FL (US)

(72) Inventors: Dinesh Srinivasan, San Jose, CA (US); Ted Stover, Hobe Sound, FL (US)

(21) Appl. No.: 18/082,128

(22) Filed: Dec. 15, 2022

Related U.S. Application Data
(60) Provisional application No. 63/290,313, filed on Dec. 16, 2021.

(51) Int. Cl. A61K 38/46

(52) U.S. Cl. CPC A61K 38/465; C12Y 301/01003

(57) ABSTRACT

Disclosed are compositions and formulations of lipase and methods of treatment and manufacture thereof.

### FIELD OF THE INVENTION

The present invention relates to stable lipase compositions and dosage forms, methods of treatment and methods of preparation.

### BACKGROUND

Lipase 2 (LIP2) from Yarrowia lipolytica (i.e., adrulipase) is an autologous yeast recombinant lipase. The targeted indication of adrulipase is the compensation of exocrine pancreatic insufficiency (EPI) due to cystic fibrosis, chronic pancreatitis and other indications the exocrine pancreas is responsible for. The symptomatology of EPI is essentially due to pancreatic lipase deficiency, an enzyme that hydrolyses triglycerides into monoglycerides and free fatty acids.

Chronic Pancreatitis (CP), the most common cause of EPI, is a long-standing inflammation of the pancreas that alters its normal structure and functions, which is associated with EPI in about 60% of patients. Cystic fibrosis (CF), another frequent aetiology of EPI, is a severe genetic disease associated with chronic morbidity and life-span decrease of most affected individuals. About 80-90% of patients with CF develop EPI. In addition, EPI is common after surgical resection of the pancreas, which is usually performed as a result of cancer or complications of CP. Other less common aetiologies of EPI include gastric surgery, certain intestinal disorders (e.g. severe celiac disease, small bowel resection, and enteral-artificial nutrition), and pancreatic diseases (e.g. pancreatic trauma, severe acute pancreatitis with pancreatic necrosis, and pancreatic cancer).

The compensation of EPI, which classically relies on porcine pancreatic extracts (PPEs, also referred to as porcine pancreatic replacement therapy (PERT)), has been a concern of the Food and Drug Administration (FDA) because of the animal ingredients used for the preparation of PPEs and the related risk of transmission of conventional and non-conventional infectious agents. In addition, the dose of PPE that can be given may be limited, especially in CF, due to the risk of fibrosing colonopathy possibly associated with the presence of proteases and/or gastro-protection agents.

Lipases in general have many pharmaceutical and industrial uses.

Accordingly, there continues to be a need in the art for pharmaceutical and industrial compositions comprising lipase such as adrulipase for the treatment of EPI.

### SUMMARY OF THE INVENTION

In certain embodiments, the present invention is directed to temperature stable lipase dosage forms, methods of treatment and methods of manufacture.

In certain embodiments, the present invention is directed to temperature stable lipase industrial compositions, methods of use and methods of manufacture.

In certain embodiments, the present invention is directed to a composition comprising (a) a lipase; and (b) one or more excipients; wherein after exposure to about a 37° C. environment for 15 minutes, the composition exhibits an activity (nmoles/min/mg) of at least 125.

In certain embodiments, the invention is directed to processes for manufacturing the compositions and dosage forms disclosed herein.

In certain embodiments, the invention is directed to methods of treating exocrine pancreatic insufficiency comprising administering a dosage form as disclosed herein. In certain embodiments the insufficiency can be caused by one or more of acute or chronic pancreatitis, cystic fibrosis, pancreatectomy (associated with or without cancer such as pancreatic cancer), age related, Shwachman-Diamond Syndrome, diabetes type 1, diabetes type 2, HIV, celiac disease, or inflammatory bowel disease (such as ulcerative colitis or Crohn’s disease).

The present invention is also directed in certain embodiments to a method of treating a condition (e.g., colon disease or other condition that is treatable by targeted administration of an active agent to the colon) by administering to a subject any of the compositions disclosed herein. The delivery can be to treat a colon disease or condition and can also be used to treat a systemic condition with a drug that is suitable for absorption in the colon.

The present invention is also directed in certain embodiments to a method of delivering an active agent to the colon of a patient by orally administering a formulation disclosed herein. In certain embodiments, at least 80%, at least 85%, at least 90%, at least 95%, or at least 99% of the active agent is delivered to the colon of the patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the effect of temperature on adrulipase activity.

FIG. 2 shows the effect of temperature on adrulipase structure.

FIG. 3 shows the effect of temperature on adrulipase activity in various formulations.

FIG. 4 shows the effect of temperature on pancrelipase (Creon®) activity.

FIG. 5 shows the effect of temperature on porcine lipase activity.

FIGS. 6 and 7 depict a schematic of the process and particle size data of Example 1.

FIG. 8 shows activity of granulated formulations and non-granulated formulations of Example 1.

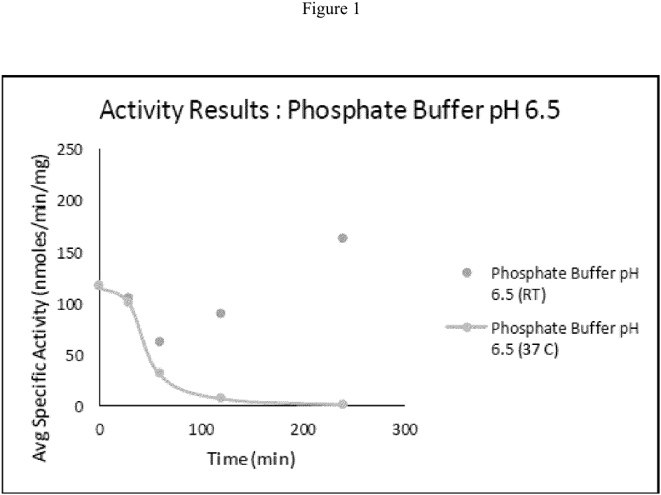

### DETAILED DESCRIPTION OF THE INVENTION

The present invention advances the state of the art by developing stable lipase compositions, pharmaceutical compositions, methods of treatment, methods of use and methods of preparation.

By virtue of the present invention, it was discovered that lipase compositions (e.g., adrulipase, pancrelipase, porcine lipase, PERT) exhibit loss of activity over time due to temperature. As set forth in the Examples, it was found that there is loss of activity at 37C (physiological temperature) with a less pronounced loss or full retention of activity at 25C (room temperature). It appeared that pH and buffer did not significantly effect this loss. This has significance in lipase pharmaceutical therapy as the active agent will quickly degrade upon introduction to physiological temperature after administration (e.g., to the gastrointestinal tract).

In certain embodiments, the present invention is directed to a pharmaceutical composition comprising (a) adrulipase; and (b) one or more pharmaceutically acceptable excipients that is stable, e.g., wherein after exposure to about a 37° C. environment for 15 minutes, the composition exhibits an activity (nmoles/min/mg) of at least 125, at least 130, at least 135, at least 140, at least 145, at least 150, at least 155 or at least 160, e.g., as measured by the assay in Example 1.

In certain embodiments, after exposure to about a 37° C. environment for 30 minutes, the adrulipase composition exhibits an activity (nmoles/min/mg) of at least 110, at least 115, at least 120, at least 125, at least 130, at least 135, or at least 140, e.g., as measured by the assay in Example 1.

In certain embodiments, after exposure to about a 37° C. environment for 45 minutes, the adrulipase composition exhibits an activity (nmoles/min/mg) of at least 100, at least 105, at least 110, at least 115, at least 120, at least 125, at least 130, at least 135, at least 140, at least 145, at least 150, or at least 155, e.g., as measured by the assay in Example 1.

In certain embodiments, after exposure to about a 37° C. environment for 90 minutes, the adrulipase composition exhibits an activity (nmoles/min/mg) of at least 75, at least 80, at least 85, at least 90, at least 95, at least 100, at least 105, at least 110, at least 115, at least 120, at least 125, or at least 130, e.g., as measured by the assay in Example 1.

In certain embodiments, the present invention is directed to a pharmaceutical composition comprising (a) a lipase (e.g.; adrulipase); and (b) one or more pharmaceutically acceptable excipients that is stable, e.g., wherein after exposure to about a 37° C. environment for 90 minutes, the composition exhibits an activity (nmoles/min/mg) that decreases by no more than 50%, no more than 40%, no more than 35%, no more than 25%, no more than 20%, no more than 10% or no more than 5%, e.g., as measured by the assay in Example 1.

In certain embodiments, the exposure is in-vivo. In other embodiments, the exposure is in-vitro.

In certain embodiments, the exposure is after oral administration to a human subject.

In certain embodiments, the lipase and the excipient are spray dried.

In certain embodiments, the lipase and the excipient are granulated and/or extruded. The granulation can be, e.g., a wet or dry granulation. Dry granulation is the process of forming grains or granules from a powdery or solid substance, producing a granular material. Typically, granulation involves agglomeration of fine particles into larger granules, e.g., to a size range (D50) between 0.01 and 3.0 mm. The agglomeration process may involve, e.g., the use of blending, applying pressure to compress the blend (roller or tablet/slug compaction), shredding or grinding solid material into finer granules or pellets and sieving. Wet granulation is very similar but will incorporate a solvent in the mixing process to generate the agglomeration and may include a drying method to remove the solvent once the granules are formed.

In certain embodiments, the lipase and the excipient are compressed.

In certain embodiments, the lipase and the excipient are spray dried and granulated and/or extruded.

In certain embodiments, the lipase and the excipient are spray dried and compressed.

In certain embodiments, the lipase and the excipient are granulated and/or extruded and compressed.

In certain embodiments, the lipase and the excipient are spray dried, granulated and/or extruded and compressed.

In certain embodiments, the lipase is a non-porcine lipase.

In certain embodiments, the lipase is porcine lipase

In certain embodiments, the non-porcine lipase is a triacylglycerol hydrolase.

In certain embodiments, the non-porcine lipase has a molecular weight of about 30 kDa to about 45 kDa.

In certain embodiments, the non-porcine lipase has a molecular weight of about 36-40 kDa or about 36 kDa, abot 37 kDa, about 39 kDa or about 40 kDa.

In certain embodiments, the non-porcine lipase contains from about 295 to about 310 amino acids.

In certain embodiments, the non-porcine lipase contains about 301 amino acids.

In certain embodiments, the non-porcine lipase is produced from Yarrowia lipolytica.

In certain embodiments, the non-porcine lipase is encoded by the LIP2 gene.

In certain embodiments, the non-porcine lipase is adrulipase.

In certain embodiments, the porcine lipase is pancrelipase.

In certain embodiments, the excipient is an oligosaccharide.

In certain embodiments, the spray drying forms particles having a D50 of about 1 micron to about 200 micron, about 1 micron to about 50 micron, 50 micron to about 150 micron, about 60 micron to about 120 micron, about 65 micron to about 85 micron or about 70 micron to about 82 micron.

In certain embodiments, the excipient comprises maltodextrin, xylan, mannan, fucoidan, galactomannan, chitosan, raffinose, stachyose, pectin, inulin, levan, graminan, and amylopectin, sucrose, lactulose, lactose, maltose, trehalose, cellobiose, nigerotriose, maltotriose, melezitose, maltotriulose, raffinose, kestose, or mixtures thereof.

In certain embodiments, the ratio of lipase to the one or more excipients is about 1:5 to about 5:1; about 1:3 to about 3:1; about 1:2 to about 2:1; about 1:1 or about 1:2.

In certain embodiments, the spray drying is performed at a pH of about 3 to about 5, about 2 to about 7 or about 6.

In certain embodiments, the spray drying is performed at an inlet temperature of greater than about 125° C. or from about 100° C. to about 250° C. or about 150° C. to about 180° C. or about 155° C. to about 165° C. or about 162° C. and/or an outlet temperature of less than about 150° C. or from about 50° C. to about 125° C. or about 60° C. to about 100° C.

In certain embodiments, the spray drying produces the lipase at a yield of greater than about 80%, greater than about 90%, greater than about 95% or greater than about 99%.

In certain embodiments, the composition comprises an enteric material.

In certain embodiments, the invention is directed to a method of treating exocrine pancreatic insufficiency comprising administering a pharmaceutical composition as disclosed herein.

In certain embodiments, the invention is directed to a method of treating acute or chronic pancreatitis comprising administering a pharmaceutical composition as disclosed herein.

In certain embodiments, the invention is directed to a method of treating cystic fibrosis comprising administering a pharmaceutical composition as disclosed herein.

In certain embodiments, the invention is directed to a method of increasing the efficacy of a lipase comprising administering a pharmaceutical composition as disclosed herein.

In certain embodiments, the insufficiency is caused by pancreatectomy such as due to pancreatic cancer.

In certain embodiments, the insufficiency is age related or due to Schachman-Diamond Syndrome, diabetes type 1, diabetes type 2, HIV, celiac disease, or inflammatory bowel disease.

In certain embodiments, the insufficiency due to ulcerative colitis or Crohn’s disease.

In certain embodiments, at least a portion of the lipase is delivered to the duodenum of the patient.

Certain embodiments of the present invention are directed to a process of preparing a pharmaceutical composition comprising combining a lipase and a pharmaceutically acceptable excipient to form a composition as disclosed herein.

### Other Embodiments

In certain embodiments, the formulations disclosed herein are delayed or immediate or sustained release oral dosage forms comprising a lipase such as a non-porcine lipase. In alternative embodiments, the dosage form comprises an enteric material encompassing or dispersed with the lipase.

In certain embodiments, the invention is directed to spray dried lipase as disclosed herein.

In certain embodiments, the formulations disclosed herein comprising a second agent that can be an additional lipase or active agent. The second agent can be selected from a fat-soluble vitamin, a protease, an amylase, a porcine pancreatic enzyme replacement, other non-porcine replacements, or a combination thereof. In certain embodiments, the vitamin is A, D, E, K or combinations thereof. In other embodiments, the second active agent is pancrelipase, liprotamase or a combination thereof.

In certain embodiments, the lipase and the second active agent are each independently immediate release, delayed release, sustained release or a combination thereof.

In certain embodiments, the dosage forms disclosed herein are contained in a capsule wherein the capsule optionally includes an enteric material, e.g., coated over the capsule or dispersed within the capsule. In other embodiments, the enteric material is spray dried with the active agent or spray dried active agent is mixed with an enteric material.

In certain embodiments, the dosage form comprises adrulipase in an amount of from about 0.5 g per day to about 10 g per day, about 2 g per day to about 5 g per day or about 2 g per day to about 4 g per day. In certain embodiments, the dosing is about 1.6 g per day, about 2.2 g per day or about 4.4 g per day.

In certain embodiments, the dosage forms disclosed herein comprise a tablet optionally comprising an enteric material, e.g., coated over the tablet or dispersed within the tablet.

In certain embodiments, the lipase (e.g., adrulipase) can be in the form of a powder optionally including an enteric material, e.g., by dry mixing, wet granulation or co-spray dried or co-freeze dried.

In certain embodiments, the formulation is a powder or particles and contained in a capsule, sachet or powder paper.

In certain embodiments, the enteric material comprises a naturally occurring material or a non-naturally occurring material.

In certain embodiments, the enteric material comprises a cellulosic material, an acrylic polymer, or a combination thereof.

In certain embodiments, the enteric material comprises hydroxypropylmethylcellulose acetate succinate.

In certain embodiments, the enteric material comprises methacrylic acid polymers, cellulose acetate phthalate polymers, hydroxypropylmethyl cellulose acetate succinate polymers, hydroxypropylmethyl cellulose phthalate polymers, polyvinyl acetate phthalate polymers or combinations thereof.

In certain embodiments, the enteric material comprises methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, shellac or combinations thereof.

In certain embodiments, the enteric material comprises hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate or combinations thereof.

In certain embodiments, the enteric material is insoluble or substantially insoluble at a pH of less than about 4, less than about 3 or less than about 2.

In certain embodiments, the enteric material does not crack, break or rupture at a pH of less than about 4, less than about 3 or less than about 2.

In certain embodiments, the enteric material is soluble or substantially soluble at a pH of greater than about 5, greater than about 5.5, greater than about 6, greater than about 7 or greater than about 8.

In certain embodiments, the enteric material cracks, breaks or ruptures at a pH of greater than about 5, greater than about 5.5, greater than about 6, greater than about 7 or greater than about 8.

In certain embodiments, the dosage forms disclosed herein release less than about 20%, less than about 18%, less than about 15%, less than about 12%, less than about 10%, less than about 5%, less than about 3% or less than about 1% lipase (e.g., adrulipase) at 30 minutes when tested in 900 mL simulated gastric fluid (at one or more points of buffer pH less than or equal to 3.0, e.g., 3.0 and/or 1.2) at 37° C. in a USP Apparatus II at 50 rpm, 75 rpm or 100 rpm with or without sinkers.

In certain embodiments, the dosage forms release less than about 10%, less than about 5%, less than about 3% or less than about 1% lipase (e.g., adrulipase) at 60 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% lipase (e.g., adrulipase) at 90 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% lipase (e.g., adrulipase) at 120 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or more of the active agents at 30 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 60 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 90 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases less than about 10%, less than about 5%, less than about 3% or less than about 1% of one or both of the active agents at 120 minutes when tested in 900 mL simulated gastric fluid (at one or more points of buffer pH less than or equal to 3.0, e.g., 3.0 and/or 1.2) at 37° C. in a USP Apparatus II at 100 rpm with or without sinkers.

In certain embodiments, the dosage form releases at least about 75%, at least about 90%, at least about 95% or at least about 99% lipase (e.g., adrulipase) at 15 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage form releases at least about 75%, at least about 90%, at least about 95% or at least about 99% lipase (e.g., adrulipase) at 30 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage form releases at least about 75%, at least about 90%, at least about 95% or at least about 99% lipase (e.g., adrulipase) at 45 minutes when tested in 900 mL simulated intestinal (at one or more points of buffer pH greater than or equal to 5.5, e.g., 5.5 or 6.0 or 6.5 or 6.6 ) at 37° C. in a USP Apparatus II at 50 rpm, 75 rpm or 100 rpm with or without sinkers.

In certain embodiments, the dosage form releases at least about 75%, at least about 90%, at least about 95% or at least about 99% lipase (e.g., adrulipase) at 60 minutes when tested in 900 mL simulated intestinal (at one or more points of buffer pH greater than or equal to 5.5, e.g., 5.5 or 6.0) at 37° C. in a USP Apparatus II at 100 rpm with or without sinkers.

In certain embodiments, the dosage forms disclosed herein target release of the lipase (e.g., adrulipase) in the duodenum of a patient in need thereof.

In certain embodiments, the lipase (e.g., adrulipase) lipase is prepared by a process comprising drying, such as freeze drying or spray drying.

In certain embodiments, the spray draying utilizes a stabilizer such as an oligosaccharide, e.g., maltodextrin.

In certain embodiments, the dried non-porcine lipase is in the form of a powder or particles. The particles can have a particle size, e.g. with a D50 of about 1 micron to about 200 micron, about 1 micron to about 50 micron, 50 micron to about 150 micron, about 60 micron to about 120 micron, about 65 micron to about 85 micron or about 70 micron to about 82 micron.

In certain embodiments, the stabilizer is maltodextrin, xylan, mannan, fucoidan, galactomannan, chitosan, raffinose, stachyose, pectin, inulin, levan, graminan, and amylopectin, sucrose, lactulose, lactose, maltose, trehalose, cellobiose, nigerotriose, maltotriose, melezitose, maltotriulose, raffinose, kestose, arginine, glycine, CaCl₂ or mixtures thereof.

In certain embodiments, the ratio of active to stabilizer is about 1:5 to about 5:1; about 1:3 to about 3:1; about 1:2 to about 2:1; about 1:1 or about 1:2.

In certain embodiments, the spray drying is performed at a pH of about 3 to about 5, about 2 to about 7, about 4 or about 6.

In certain embodiments, the spray drying is performed at a temperature of greater than about 125° C., greater than about 150° C., or from about 100° C. to about 250° C. or about 150° C. to about 180° C. or about 155° C. to about 165° C.

In certain embodiments, the spray drying produces the non-porcine lipase at a yield of greater than about 80%, greater than about 90%, greater than about 95% or greater than about 99%.

In certain embodiments, the methods of treatment are solely with the non-porcine lipase formulations disclosed herein without the concurrent administration of a second active agent such as a fat-soluble vitamin (e.g., vitamin A, D, E, K and combinations thereof), a protease, an amylase, a porcine pancreatic enzyme replacement, other non-porcine replacements, pancrelipase, liprotamase a combination of three enzymes: lipase, protease, and amylase or a combination thereof.

In certain embodiments, the dosage form is administered by feeding tube in the form of a solution or suspension or sprinkled on food in the form of a powder or administered as an oral dosage form such as a capsule, powder, tablet, liquid or semi-solid.

In certain methods disclosed herein, at least a portion of the lipase (e.g., adrulipase) is delivered to the duodenum of the patient. The portion can be, e.g., at least about 75%, at least about 85%, or at least about 95%.

In certain embodiments, the present formulations and methods provide (i.e. after treatment) a CFA% in individual patients or subjects from about 70 to about 99, about 75 to about 98, about 80 to about 92, about 85 to about 92, about 86 to about 92 or about 90 to about 92. In certain embodiments, the patient to be treated (i.e., prior to treatment) has a CFA% in individual patients or subjects of less than about 80, less than about 60, less than about 40, from about 15 to about 80 or about 30 to about 60.

In certain embodiments, the present formulations and methods provide a CNA% in individual patients or subjects from about 90 to about 99, about 92 to about 99, about 95 to about 99 or about 99 to about 99.

In certain embodiments, the present formulations and methods provide a CNA% in a population of patients or subjects from about 90 to about 99, about 92 to about 99, about 95 to about 99 or about 99 to about 99.

In certain embodiments, the present formulations and methods provide a CFA gain relative to mean of about 3% to about 12%, from about 4% to about 10%, about 2% to about 6%, about 3% to about 6%, about 4%, about 5% or about 6%.

In certain embodiments, the present formulations and methods provide a maximum individual relative CFA gain from about 5% to about 50%, about 10% to about 45%, about 15% to about 40%, about 20% to about 50%, about 30% to about 40%, about 30%, about 35% or about 40%.

In certain embodiments, the invention is directed to preparing the compositions and formulations disclosed herein.

In other embodiments, the compositions disclosed herein are utilized for industrial methods and uses, e.g., biocatalysts; detergents; food; environmental industries; production of citric acid and aroma from a variety of carbon sources, including sugars, alkanes, plant oils, starch hydrolysates, ethanol, and glycerol; degradation of hydrophobic substrates; degradation of organic compounds; bioremediation of environments contaminated with oil spills, aliphatic and aromatic compounds, organic pollutants, 2,4,6-trinitrotoluene, and metals; and processes for the synthesis of beta-hydroxy butyrate, L-dopa, and emulsifiers

In certain embodiments, the non-porcine lipase is the secreted acid-resistant lipase (LIP2) from the yeast Yarrowia lipolytica. It belongs to the family of triacylglycerol lipases. It shares the common fold of a/b hydrolases and the crystal structure has been solved.

LIP2 is a 301 amino acid protein, which is secreted in culture medium as a glycosylated mature form after cleavage of a 39 amino acid signal peptide. Alternative cleavages have been evidenced on the lipase resulting in N-terminal sequence heterogeneity. The main N-terminal sequence was identified as STETSHIDQESYNFF in the spray-dried powder. This protein is also referred to as MS1819 or adrulipase.

Two N-glycosylation sites have been identified at residues N113 and N134 and analysis by mass spectrometry has revealed that each site harbors the following sugar moieties GlcNAc2-Manx (x=8). Five major glycoforms have been evidenced by isoelectrofocusing gels (IEF). In certain embodiments, the drug substance is defined as the spray-dried active agent bulk solution following the addition of maltodextrine.g., in a ratio of 1:3 to 3:1 or 2:1 (based on bulk dry matter weight). In other embodiments the drug substance is defined as active agent prepared by spray drying with maltodextrin in a ratio of 1:3 or 3:1 or 2:1 and an enteric polymer (e.g., HPMCAS) in a ratio of 1:10 to 10:1 or about 5:1. The active substance can also have 1-15% salts.

The sequence is as follows (SEQ ID NO.1):

VYTSTETSHIDQESYNFFEKYARLANIGYCVGPGTKIFKPFNCGLQCAHFPNVELIEEFH
DPRLIFDVSGYLAVDHASKQIYLVIRGTHSLEDVITDIRIMQAPLTNFDLAANISSTATC
DDCLVHNGFIQSYNNTYNQIGPKLDSVIEQYPDYQIAVTGHSLGGAAALLFGINLKVN
GHDPLVVTLGQPIVGNAGFANWVDKLFFGQENPDVSKVSKDRKLYRITHRGDIVPQVP
FWDGYQHCSGEVFIDWPLIHPPLSNVVMCQGQSNKQCSAGNTLLQQVNVIGNHLQYF
VTEGVCGI.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLE 1

Adrulipase powder was prepared by spray drying (w/w%): 54.4% adrulipase : 27% maltodextrin : 10% HPMCAS : 8.4 % Salts. Other embodiments can have 40% to 75% adrulipase, 10-40% maltodextrin, 5-20% HPMCAS and 1-15% salts.

A granulation was prepared with the adrulipase spray dried powder (69%) microcrystalline cellulose (30%; 10% x 3) and 1% magnesium stearate.

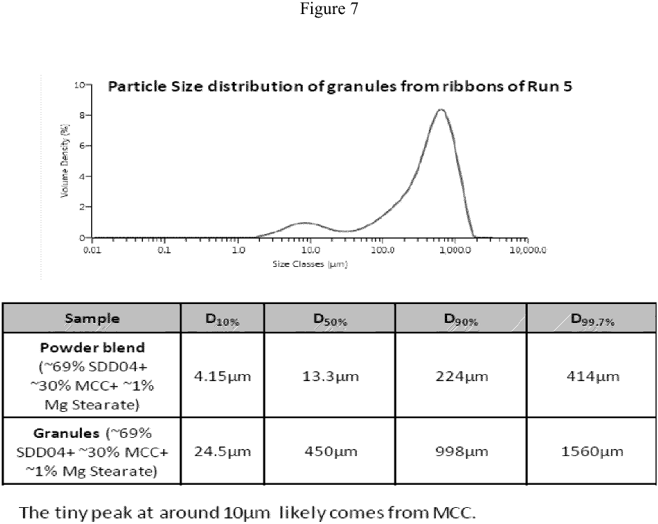

The microcrystalline cellulose was added in three portions followed by geometric mixing for 5 minutes and the mixture was sieved through 425 micron mesh and then pressed into 100 mg tablets at 1000 psi followed by granulation through 850 micron mesh to provide powder with a D50 of 111 microgram. Another portion was pressed into 200 mg tablets at 2000 psi followed by granulation through 850 micron mesh to provide powder with a D50 of 455 micron. A schematic of the process and particle size data are presented in FIGS. 7A and 7B.

Activity of the spray dried powder and the granules was tested according to the following procedure:

### Activity Method

Details of the activity method we used to generate all the formulation development data we have been discussing:
Colorimetric Activity Assay (96 well)
Scope: Adrulipase is a lipase which hydrolyzes DMPTB to release a free thiol group.
   This thiol group reacts with DTNB (Ellman’s reagent) to produce TNB (colored product).
   Production of TNB is quantified using a standard curve and the values are used to calculate the enzymatic activity of Adrulipase.
      DMPTB 2,3-Dimercapto-1-propanol tributyrate
      DTNB 5.5′- dithiobis(2-nitrobenzoic acid)
      TNB 2-Nitro-5-thiobenzoic acid
Sample Prep
   Prepare 0.5 mg/mL lipase solution in 50 mM Tris
   Mix 50 µg of lipase (from above) with 0.05% Triton X-100, 0.6 mM DMPTB and 0.8 mM DTNB - begin measuring immediately
Analysis
   Wavelength: 412 nm
   Type/mode of analysis: Kinetic
   Total Duration: 30 mins, with a reading taken every 30s (61 readings)
   Plot the linearity curve of TNB and obtain the slope and intercept values to calculate activity

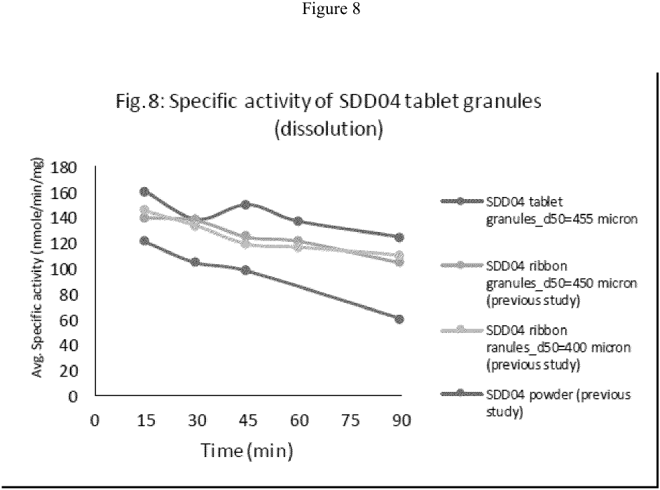

The results are set forth in FIG. 8.

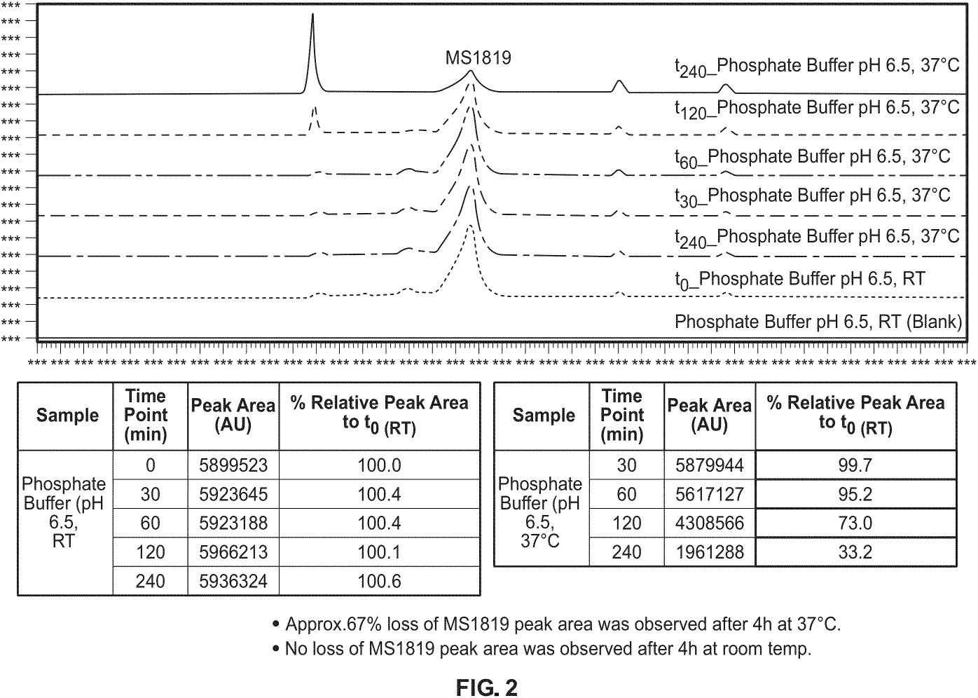

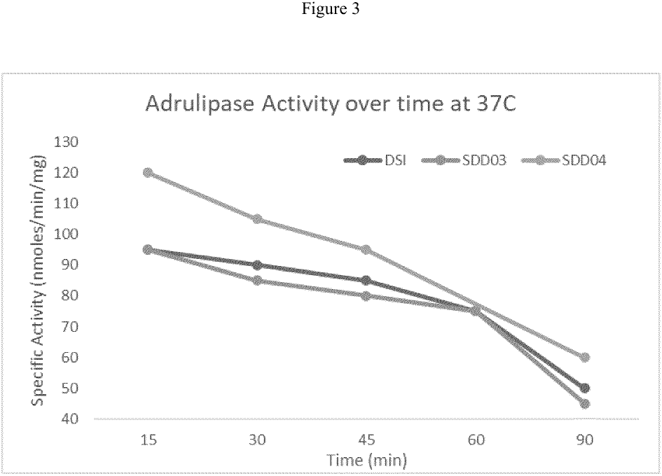

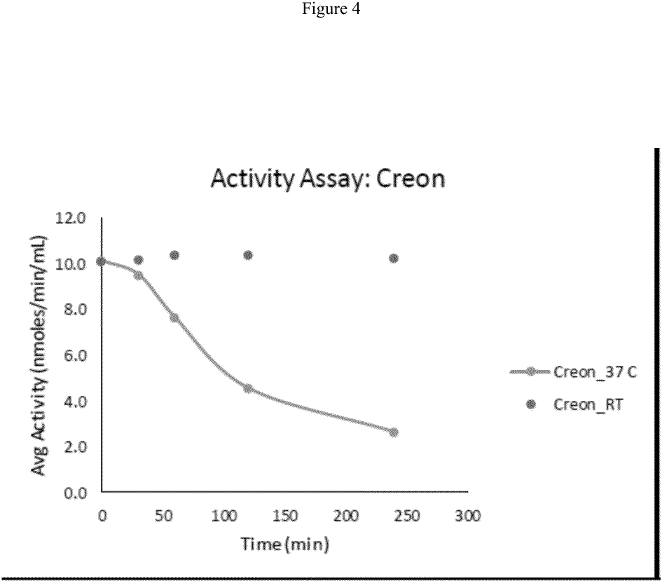

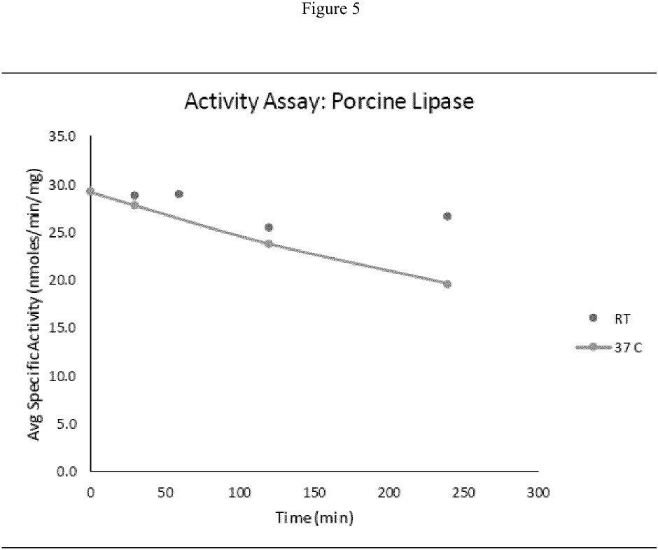

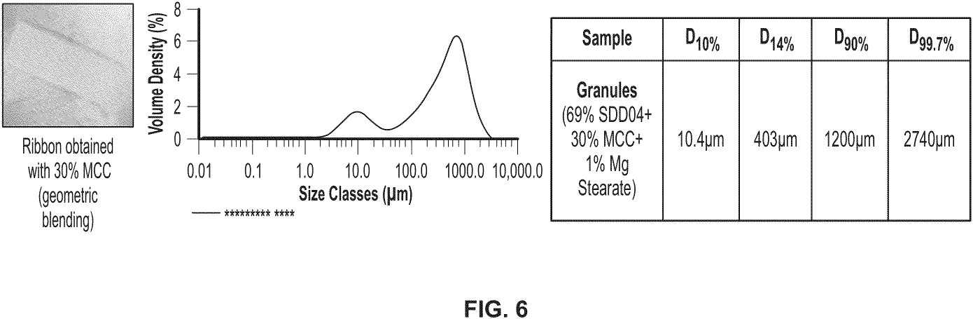

FIG. 2 shows the effect of temperature on adrulipase structure utilizing size exclusion chromatography (SEC). Details of the SEC method is set forth below:
High Performance Liquid Chromatography (HPLC) method
Mobile Phase
   20 mM Phosphate / 100 mM NaCl, pH 6
Sample Prep
   Prepare 1.5 mg/mL lipase solution in Mobile Phase
Analysis
   Column: TSKgel G2000SW_{XL}
   Flow rate: 0.5ml/min
   Column Temp: 20° C.
   Wavelength: 280 nm
   Run time: 40 mins
   Calculate purity based on total peak area in the chromatogram
   Calculate Assay by peak area comparison to a reference standard

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims

1. A pharmaceutical composition comprising:
(a) a lipase; and
(b) one or more pharmaceutically acceptable excipients;
wherein after exposure to about a 37° C. environment for 15 minutes, the composition exhibits an average specific activity (nmoles/min/mg) of at least 75.

2-4. (canceled)

5. The pharmaceutical composition of any of claim 1 , wherein the exposure is in-vivo.

6. The pharmaceutical composition of claim 5, wherein the exposure after oral administration to a human subject.

7. The pharmaceutical composition of claim 1, wherein the lipase and the excipient are spray dried.

8. The pharmaceutical composition of claim 1, wherein the lipase and the excipient are granulated.

9. The pharmaceutical composition of claim 1, wherein the lipase and the excipient are compressed.

10. The pharmaceutical composition of claim 1, wherein the lipase and the excipient are spray dried and granulated.

11. The pharmaceutical composition of claim 1, wherein the lipase and the excipient are spray dried and compressed.

12. The pharmaceutical composition of claim 1, wherein the lipase and the excipient are granulated and compressed.

13. The pharmaceutical composition of claim 1, wherein the lipase and the excipient are spray dried, granulated and compressed.

14. The pharmaceutical composition of claim 1, wherein the lipase is a non-porcine lipase.

15. The pharmaceutical composition of claim 1, wherein the lipase is porcine lipase.

16. The pharmaceutical composition of claim 14, wherein the non-porcine lipase is a triacylglycerol hydrolase.

17. The pharmaceutical composition claim 14, wherein the non-porcine lipase has a molecular weight of about 30 kDa to about 45 kDa.

18-32. (canceled)

33. A method of treating exocrine pancreatic insufficiency comprising administering a pharmaceutical composition according to claim 1.

34. A method of treating acute or chronic pancreatitis comprising administering a pharmaceutical composition according to claim 1.

35. A method of treating cystic fibrosis comprising administering a pharmaceutical composition according to claim 1.

36. A method of increasing the efficacy of a lipase comprising administering a pharmaceutical composition according to claim 1.

37. The method of claim 33, wherein the insufficiency is caused by pancreatectomy such as due to pancreatic cancer.

38. The method of claim 33, wherein the insufficiency is age related or due to Schachman-Diamond Syndrome, diabetes type 1, diabetes type 2, HIV, celiac disease, or inflammatory bowel disease.

39-42. (canceled)
